SEQUENCE LISTING (6 sequences)

SEQ ID NO: 1 - DNA, 24 bp, Artificial Sequence
  Synthetic primer
tgcacagagagaaatacccgaana

SEQ ID NO: 2 - DNA, 17 bp, Artificial Sequence
  Synthetic primer
tgcancttcggcagcat

SEQ ID NO: 3 - DNA, 26 bp, Artificial Sequence
  Synthetic primer
aagtatcgacctcgtcggaaggcgaa

SEQ ID NO: 4 - DNA, 22 bp, Artificial Sequence
  Synthetic primer
gtgcacctgactcctgaggaga

SEQ ID NO: 5 - DNA, 21 bp, Artificial Sequence
  Synthetic primer
ccttgataccaacctgcccag

SEQ ID NO: 6 - DNA, 26 bp, Artificial Sequence
  Synthetic primer
aaggtgaacgtggatgaagttggtgg

(19) United States
(12) Patent Application Publication
(10) Pub. No.: US 2012/0058480 A1
(43) Pub. Date: Mar. 8, 2012

(54) ANTIGENIC APPROACH TO THE DETECTION AND ISOLATION OF MICROPARTICLES ASSOCIATED WITH FETAL DNA

(76) Inventors: Dorothy E. Lewis, Houston, TX (US); Aaron F. Orozco, Houston, TX (US); Farideh Z. Bischoff, Sugar land, TX (US)

(21) Appl. No.: 13/203,031

(22) PCT Filed: Feb. 24, 2010
(86) PCT No.: PCT/US10/25209

§ 371 Date: Aug. 24, 2011

Related U.S. Application Data
(60) Provisional application No. 61/155,094, filed on Feb. 24, 2009.

(51) Int. Cl. C12Q 1/68; C09K 3/00; C12P 19/34; C12N 9/96; C07H 21/04; C07H 1/08

(52) U.S. Cl. 435/6.12; 536/25.4; 435/91.2; 435/188; 252/183.11

(57) ABSTRACT

The present disclosure provides methods of quantifying and isolating microparticles of fetal from maternal bodily fluids such as cell free maternal plasma. Antibodies or combinations of antibodies that selectively bind fetal microparticles in maternal bodily fluid permit quantification by flow cytometry and isolation through flow cytometry based sorting and immunopurification.

### CROSS-REFERENCE TO RELATED APPLICATIONS

This application claims priority to, and for the U.S. the benefit under 35 U.S.C. §119(e) of, U.S. Provisional Patent Application 61/155,094, filed 24 Feb. 2009. The contents of this priority application are hereby incorporated herein in their entirety.

### STATEMENT REGARDING FEDERALLY SPONSORED RESEARCH OR DEVELOPMENT

This invention was made with government support under HD R01-046623 and T32AI007495 awarded by NIH/NICHD and NIAID. The U.S. Federal Government has certain rights in the invention.

### TECHNICAL FIELD

The technical field of the disclosure is the field of physical and chemical analysis of biologic materials corresponding generally to IPC G01N 33/483 and G01N 33/50.

### BACKGROUND OF THE INVENTION

Early intervention in pregnancy, that ranges from maternal nutrition to fetal surgery offer parents many new opportunities to help unborn children achieve optimal health in utero and after birth. In addition, advanced notice of medical conditions allows parents to plan for helping children born with certain medical conditions. Prenatal diagnosis of aneuploidy and single-gene disorders can significantly enhance outcomes by providing parents and their medical providers addition medical information. Unfortunately, current means of prenatal genetic testing involve methods such as amniocentesis and chorionic villous sampling (CVS). These procedures are associated with significant risk to both fetus and mother. Therefore, alternative methods for safely obtaining fetal genetic material are necessary if fetal medicine is to fully benefit from available genetic testing.

Analysis of cell free fetal DNA (cffDNA) in maternal plasma offers one potential approach to non-invasive prenatal diagnosis. cffDNA has been used for non-invasive rhesus D typing, sex determination and detection of a few genetic mutations such as achondroplasia. In addition, elevated levels of cffDNA are observed in some chromosomal aneuploidies such as Trisomy 21.

cffDNA has limitations, primarily because cell free maternal DNA is also present in blood in quantitative excess (approximately 96%). The background of excess maternal origin DNA generally precludes genetic testing cffDNA for single nucleotide polymorphisms (SNPs) and is technically challenging for many other types of testing such as microsatellite based gene tests.

Because total cell free DNA from maternal blood has limited uses, attempts have been made to enrich specifically for the cffDNA. One such approach has been to take advantage of the nucleosomal histone proteins associated with some cffDNA. WO/2007/121276 ENRICHMENT OF CIRCULATING FETAL DNA. Fetal DNA may be enriched using antibodies to the histone H3.1 enriched in fetal DNA as well as more accessible in cffDNA versus the background maternal DNA. Other methods of cffDNA enrichment or analysis include gel electrophoresis separation as well as a Mass Spectrometry technique. An alternative source of fetal DNA is intact fetal cells isolated from maternal circulation. Fetal cells are shed into maternal circulation in very small numbers and represent either differentiating cells, such as nucleated red blood cells or trophoblastic cells, which might be multinucleate, confusing genetic analysis. Consequently, isolation of fetal cells is expensive and labor intensive.

Although enrichment processes for total cffDNA and fetal cells represent significant advances, improved means of isolating DNA of fetal origin from maternal bodily fluids is still needed. In particular, there is a need for a technically simple and robust way to enrich for fetal DNA from maternal bodily fluids for use in routine clinical diagnostics.

A significant portion of fetal DNA in maternal bodily fluids is present in the form of subcellular microparticles in the size range of 0.5-3 micrometers. The methods herein are directed to quantifying, enriching and isolating fetal origin microparticles from maternal bodily fluids some of which contain fetal DNA.

### BRIEF SUMMARY OF THE INVENTION

The present invention provides methods for enriching fetal nucleic acids from a maternal sample, such as, whole blood, plasma, serum, urine, or mucus obtained from a pregnant female. The methods of the invention enrich fetal microparticles (membrane bound bodies arising from fetal tissue, trophoblasts, and placenta tissue, collectively referred to herein as fetal microparticles) which contain fetal nucleic acids. In one embodiment of the invention, fetal microparticles are enriched by combining the maternal sample with an antibody or ligand specific to a fetal protein or antigen present in the fetal microparticle, such that the antibody or ligand can bind to the fetal protein or antigen. The antibody-antigen or ligand-protein complex is then separated from the maternal sample enriching the fetal nucleic acids. In another embodiment of the invention, the antibody-antigen or ligand-protein complex is separated from the maternal sample by a magnetic interacting material that binds to the antibody-antigen or ligand-protein complex, and applying a magnetic field to the mixture to separate the magnetic material-antibody/ligand-fetal microparticle complex from the maternal sample.

In one embodiment, the magnetic material is a magnetic nanoparticle coated with dextran, and the magnetic material-antibody/ligand-fetal microparticle complex comprises the magnetic nanoparticle coated with dextran, an anti-dextran antibody, the antibody specific to a fetal antigen, a coupling agent to connect the anti-dextran antibody to the anti-fetal antigen antibody, and a microparticle with the fetal antigen.

In another embodiment, the anti-fetal antigen antibody is associated with a fluorescent tag, and the fluorescent tag-anti-fetal antigen antibody-fetal microparticle complex is separated from the maternal sample by flow cytometry, fluorescent activated sorting. In still another embodiment, the anti-fetal antigen antibodies are anti-HLA-G, anti-CD49e, and anti-CD51, and microparticles are enriched using polychromatic flow cytometry, fluorescent activated sorting to isolate microparticles that are CD49e+, CD51+ and HLA-G+.

In an embodiment of the invention, the above described methods are used to enrich the fetal nucleic acids relative to maternal nucleic acids in the maternal sample by, or at least by: 5, 10, 15, 20, 25, or 26 fold. In another embodiment, the fetal nucleic acids are enriched by 5-10, 5-20, 5-26, 10-20, 10-26, or 20-26 fold. The present invention also provides novel compositions of these enriched fetal microparticles, and compositions of enriched fetal nucleic acids obtained from the microparticles.

The maternal samples to be used in the invention can be any sample obtained from a pregnant female that contains fetal microparticles, for example, maternal whole blood, maternal plasma, maternal serum, maternal cervical mucus, amniotic fluid, or maternal urine. In one embodiment, the maternal sample is whole blood or derived from whole blood obtained from the pregnant female in the first or second trimester.

The fetal antigen or protein of the invention can be any protein or antigen that is preferably present or reactive with the antibody or ligand on fetal microparticles compared to maternal microparticles. In one embodiment, the fetal protein or antigen is HLA-G and the antibody is MEM-G/1 or 2A12. In another embodiment, the fetal protein or antigen is human placental alkaline phosphatase and the antibody is H17E2.

The fetal nucleic acids enriched by the methods of the invention can be used to perform prenatal diagnostics, including for example, directly sequencing or amplifying a diagnostic portion of the fetal nucleic acids to determine the genotype of the fetus. In one embodiment, the amplified, sequenced or detected nucleic acids of the fetal nucleic acids are correlated with Cystic Fibrosis, or RhD type, or sex, or Fragile-X Syndrome, or Sickle Cell Anemia, or Tay-Sachs Disease, or Thalassemia, or a chromosomal aneuploidy, e.g., Down's Syndrome, or other genetic diseases or genotype traits.

In another embodiment, the fetal nucleic acids of the invention are directly sequenced and the distance from oligonucleotide primer to the sequence of interest is less than or equal to 360 bps, preferably less than or equal to 180 bps, 150 bps, 120 bps, 100 bps, 70 bps or 50 bps; b) if amplifying by PCR, the length of the amplified sequence is less than or equal to 360 bps, preferably less than or equal to 180 bps, 150 bps, 120 bps, 100 bps, 70 bps or 50 bps.

Certain aspects of the invention are also described by the following numbered sentences:
1. A method of enriching microparticles of fetal origin having fetal DNA from a maternal bodily fluid, the method comprising the steps of
   a) combining in a volume of liquid
      i) an antibody to a fetal specific protein having antigens reactive with the antibody and
      ii) a microparticle of fetal origin,
   b) forming an immunocomplex comprising the microparticles and the antibody, and
   c) isolating the immunocomplex from the volume of liquid.
2. The method of sentence 1 wherein step b) further comprises forming an immunocomplex comprising a magnetically interacting material and wherein step c) comprises isolating the immunocomplex by application of a magnetic field to at least a portion of the volume of liquid.
3. The method of sentences 1-2, wherein the volume of liquid comprises cell free maternal plasma and/or maternal urine.
4. The method of sentences 1-3, wherein at least one fetal specific protein is an HLA-G having an epitope such as the HLA-G epitope recognized by mAb MEM-G/1 or by mAb 2A12.
5. The method of sentences 1-3, wherein at least one fetal specific protein is a human placental alkaline phosphatase having an epitope such as the epitope recognized by mAb H17E2.
6. The method of sentence 4, wherein at least one antibody is MEM-G/1 or 2A12.
7. The method of sentences 4 or 6, wherein the volume of liquid comprises cell free maternal plasma representing a first or second trimester pregnancy.
8. The method of sentence 5, wherein at least one antibody is H17E2.
9. The method of sentences 5 or 8, wherein the volume of liquid comprises cell free maternal plasma representing a first or second trimester pregnancy.
10. The method of sentences 2-9, wherein the magnetically interacting material is a nanoparticle coated with an antibody target, such as dextran, and wherein the immunocomplex comprises a tetrameric antibody complex comprising
   i) an antibody to the nanoparticle coat,
   ii) the antibody to the fetal specific protein, and
   iii) an antibody which binds to both the antibody to the nanoparticle coat and the antibody to the fetal specific protein.
11. The method of sentence 1, wherein the antibody to the fetal specific protein comprises a fluorescent tag and step c) comprises isolating the immunocomplex by flow cytometry.
12. The method of sentence 11, wherein the fetal specific protein is HLA-G, step b) further comprises forming an immunocomplex comprising the microparticles and fluorescently labeled anti-CD49e and anti-CD51 antibodies, and step c) comprises polychromatic flow cytometry base sorting to isolate microparticles labeled CD49e+, CD51+ and HLA-G+.
13. The method of sentences 1-12, further comprising the step of isolating the fetal origin DNA associated with the microparticle of fetal origin, optionally enriching the fetal origin DNA relative to maternal DNA by, or at least by, or no more than: 5, 10, 15, 20, 25 or 26 fold.
14. The method of sentence 13, further comprising the step of directly sequencing or amplifying a portion of the DNA associated with the microparticles of fetal origin.
15. The method of sentence 14 wherein the amplified or sequenced portion is indicative of the presence or absence of an inherited trait such as those indicated by the nonexhaustive list of commonly used genetic testing in the Disclosure below.
16. The method of sentences 14 and 15, wherein a) if directly sequencing, the distance from oligonucleotide primer to the sequence of interest is less than or equal to 360 bps, preferably less than or equal to 180 bps, 150 bps, 120 bps, 100 bps, 70 bps or 50 bps; b) if amplifying by PCR, the length of the amplified sequence is less than or equal to 360 bps, preferably less than or equal to 180 bps, 150 bps, 120 bps, 100 bps, 70 bps or 50 bps.
   Certain aspects of the invention are also described by the following second set of numbered sentences:
1. A method of enriching microparticles of fetal origin having fetal nucleic acids from a maternal sample, the method comprising the steps of
   a) combining in a volume of liquid
      i) an antibody that binds to a fetal specific antigen and
      ii) a microparticle of fetal origin, wherein the microparticle includes the fetal specific antigen,
   b) forming an immunocomplex comprising the microparticles and the antibody, and
   c) isolating the immunocomplex from the volume of liquid.
2. The method of sentence 1 wherein step b) further comprises forming an immunocomplex comprising a magnetically interacting material and wherein step c) comprises isolating the immunocomplex by application of a magnetic field to at least a portion of the volume of liquid.
3. The method of any one of sentences 1-2, wherein the volume of liquid comprises cell free maternal plasma and/or maternal urine.
4. The method of any one of sentences 1-3, wherein at least one fetal specific antigen is an HLA-G having an epitope such as the HLA-G epitope recognized by mAb MEM-G/1 or by mAb 2A12.
5. The method of any one of sentences 1-3, wherein at least one fetal specific protein is a human placental alkaline phosphatase having an epitope such as the human placental alkaline phosphatase epitope recognized by mAb H17E2.
6. The method of sentence 4, wherein at least one antibody is MEM-G/1 or 2A12.
7. The method of sentences 4 or 6, wherein the volume of liquid comprises cell free maternal plasma representing a first or second trimester pregnancy.
8. The method of sentence 5, wherein at least one antibody is H17E2.
9. The method of sentences 5 or 8, wherein the volume of liquid comprises cell free maternal plasma representing a first or second trimester pregnancy.
10. The method of any one of sentences 2-9, wherein the magnetically interacting material is a nanoparticle coated with an antibody target, such as dextran, and wherein the immunocomplex comprises a tetrameric antibody complex comprising
   a) an antibody to the nanoparticle coat,
   b) the antibody to a fetal specific protein, and
   c) an antibody which binds to both the antibody to the nanoparticle coat and the antibody to the fetal specific protein.
11. The method of sentence 1, wherein the antibody to the fetal specific protein comprises a fluorescent tag and step c) comprises isolating the immunocomplex by flow cytometry, fluorescent activated sorting.
12. The method of sentence 11, wherein the fetal specific protein is HLA-G, step b) further comprises forming an immunocomplex comprising the microparticles and fluorescently labeled anti-CD49e and anti-CD51 antibodies, and step c) comprises polychromatic flow cytometry, fluorescent activated sorting to isolate microparticles labeled CD49e+, CD51+ and HLA-G+.
13. The method of any one of sentences 1-12, further comprising the step of isolating the fetal origin nucleic acid associated with the microparticle of fetal origin.
14. The method of sentence 13, further comprising the step of directly sequencing or amplifying a portion of the nucleic acid associated with the microparticle of fetal origin.
15. The method of sentence 14 wherein the amplified of sequenced portion is indicative of the presence or absence of an inherited trait.
16. A method of enriching microparticles of fetal origin having fetal nucleic acids from a maternal sample, the method comprising the steps of
   a) combining in a volume of liquid
      i) an antibody that binds to a fetal specific antigen and
      ii) a microparticle of fetal origin, wherein the microparticle includes the fetal specific antigen,
   b) forming an immunocomplex comprising the microparticles and the antibody, and
   c) enriching the immunocomplex.
17. A method of detecting a fetal nucleic acid, the method comprising the steps of:
   a) enriching a microparticle of fetal origin having a fetal nucleic acid from a maternal sample by combining in a volume of liquid an antibody that binds to a fetal specific antigen and a microparticle of fetal origin, wherein the microparticle includes the fetal specific antigen, wherein the microparticle and the antibody form an immunocomplex; and
   b) performing a diagnostic test on the microparticle.
18. The method of sentence 17 further comprising the step of:
   c) performing a diagnostic test on the fetal nucleic acid within the microparticle.
      The method of any one of the preceding sentences 1-16 and 1-18 further comprising one or more further purifications of the microparticles.
      The methods and compositions substantially as described herein.

The foregoing has outlined the features and technical advantages of the present invention so that the detailed description of the invention may be better understood. Additional features and advantages of the invention will be described hereinafter which also form the subject of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1-(A-B) Half a million HTR-8/SVneo microparticles were directly labeled with PE-conjugated secondary goat anti-rabbit (GAR) F(ab′)2 fragments only or directly labeled with PE-conjugated secondary goat anti-mouse (GAM) Abs only. The results are shown as the mean±standard deviation. (A) The line graph represents the percentage of PE positive. Microparticles (n=3). (B) The line graph represents the mean fluorescence intensity (MFI) of PE positive Microparticles (n=3). (C-D) Half a million HTR-8/SVneo Microparticles were indirectly labeled with rabbit anti-human AT1 Abs (sc-1173), followed by secondary labeling using PE-conjugated GAR F(ab′)2 fragments or indirectly labeled with mouse anti-human AT1 Abs (LS-c20633), followed by secondary labeling using PE-conjugated GAM Abs and analyzed by flow cytometry. The results are shown as the mean±standard deviation. (C) The line graph represents the percentage of AT1⁺ Microparticles labeled with sc-1173 or LS-c20633 Abs (n=3). (D) The line graph represents the mean fluorescence intensity (MFI) of AT1 Microparticles (n=3). (*) indicates significant difference (P<0.05) compared to LS-C20633. (E-F) Frozen plasma samples were thawed and quantitated using the fluorescence bead-based method. One million plasma Microparticles were indirectly labeled with rabbit anti-human AT1 Abs (sc-1173), followed by secondary labeling using PE-conjugated GAR F(ab′)2 fragments and analyzed by flow cytometry. The results are shown as the mean±standard deviation. (E) The line graph represents the percentage of AT1⁺ plasma Microparticles (n=3). (F) The line graph represents the mean fluorescence intensity (MFI) of AT1 plasma Microparticles (n=3).

### DETAILED DESCRIPTION OF THE INVENTION

The disclosure and claims should be read with reference to the following definitions:

“A or An” means one or more unless it is apparent from the context that the object is singular.

“Amplifying” means increasing the number of DNA molecules having a specific sequence. The common means for amplifying DNA is PCR. However the term amplify encompasses any know technique in the art such as ligase chain reaction and cloning into high copy number plasmids.

“Antibody” means a protein functionally defined as a binding protein and structurally defined as comprising an amino acid sequence that is recognized by one of skill in the art as having variable and constant regions. A typical antibody structural unit is known to comprise a tetramer. Each tetramer is composed of two identical pairs of polypeptide chains, each pair having one “light” and one “heavy” chain. The N-terminal portion of each chain defines the variable region of about 100 to about 110 amino acids, which are primarily responsible for antigen recognition and binding. The terms variable heavy chain (V_{H}) and variable light chain (V_{L}) regions refer to these light and heavy chains, respectively. The variable region includes the segments of Framework 1 (FR1), CDR1, Framework 2 (FR2), CDR2, Framework 3, CDR3 and Framework 4 (FR4). Antibodies are typically divided into five major classes, IgM, IgG, IgA, IgD, and IgE, based on their constant region structure and immune function. The constant region is identical in all antibodies of the same isotype, but differs in antibodies of different isotypes. Heavy chains γ, α and δ have a constant region composed of three tandem (in a line) Ig domains, and a hinge region for added flexibility; heavy chains μ and ε have a constant region composed of four immunoglobulin domains. Antibody classes can also be divided into subclasses, for example, there are four IgG subclasses IgG1, IgG2, IgG3 and IgG4. The structural characteristics that distinguish these subclasses from each other are known to those of skill in the art and can include the size of the hinge region and the number and position of the interchain disulfide bonds between the heavy chains. The constant region also determines the mechanism used to destroy the bound antigen. A light chain has two successive regions: one constant region, which are designated as κ and λ, and one variable region.

“Antibody” also includes grafted antibodies. Grafted when used in reference to heavy or light chain polypeptides, or functional fragments thereof, is intended to refer to a heavy or light chain, or functional fragment thereof, having substantially the same heavy or light chain CDR of a donor antibody, respectively, absent the substitution of conservative or alternative amino acid residues outside of the CDRs as known in the art. Grafted antibodies, also known in the art as humanized antibodies, typically are human immunoglobulins (recipient antibody) which have residues from the CDR of the recipient replaced with residues from the CDR of a donor antibody, which is typically from a non-human species such as mouse, rat, rabbit or non-human primates. The donor antibodies have the desired specificity, affinity and capacity towards the target antigen. In some aspects, human framework region residues are replaced by a counterpart non-human residue. In other aspects, the grafted antibodies may have residues which are not present in either the donor or recipient antibodies. When used in reference to a functional fragment, not all donor CDRs need to be represented. Rather, only those CDRs that would normally be present in the antibody portion that corresponds to the functional fragment are intended to be referenced as the donor CDR amino acid sequences in the functional fragment. Additionally, a grafted antibody optionally will have at least a portion of an immunoglobulin constant region typical of a human immunoglobulin. Grafting techniques are well known to one of skill in the art and are reviewed in Vaswani and Hamilton, Ann. Allergy, Asthma & Immunol. 1: 105-115 (1998); Harris, Biochem. Soc. Transactions 23:1035-1038 (1995); Hurle and Gross, Curr. Op. Biotech. 5:428-433 (1994) and Brekke and Sandlie, Nature Reviews 2:52-62 (2003).

The regions between the CDRs in the variable region are called the framework (FR) regions. The FR regions typically exhibit far less variation than the CDR regions. Based on similarities and differences in the framework regions the immunoglobulin heavy and light chain variable regions can be divided into groups and subgroups.

A “CDR” refers to a region containing one of three hypervariable loops (H1, H2 or H3) within the non-framework region of the immunoglobulin (Ig or antibody) V_{H} β-sheet framework, or a region containing one of three hypervariable loops (L1, L2 or L3) within the non-framework region of the antibody V_{L} β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved β-sheet framework, and thus are able to adapt different conformations (Chothia and Lesk, J. Mol. Biol. 196:901-917 (1987)). Both terminologies are well recognized in the art. The positions of CDRs within a canonical antibody variable domain have been determined by comparison of numerous structures (Al-Lazikani et al., J. Mol. Biol. 273:927-948 (1997); Morea et al., Methods 20:267-279 (2000)). Because the number of residues within a loop varies in different antibodies, additional loop residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

“Antibody” also includes active antibody fragments, such as chemically, enzymatically, or recombinantly produced Fab fragments, F(ab)₂ fragments, or peptide fragments comprising at least one complementarity determining region (CDR) specific for a GPVI polypeptide, peptide, or naturally-occurring variant thereof. Affinities of binding partners or antibodies may be readily determined using conventional techniques, for example, by measuring the saturation binding isotherms of ¹²⁵I-labeled IgG or its fragments, or by homologous displacement of ¹²⁵I-labeled IgG by unlabeled IgG using nonlinear-regression analysis as described by Motulsky, in Analyzing Data with GraphPad Prism (1999), GraphPad Software Inc., San Diego, Calif. Other techniques are known in the art, for example, those described by Scatchard et al., Ann. NY Acad. Sci., 51:660 (1949).

In one aspect, the antibody, or functional fragment thereof is monoclonal. In another aspect, the antibody, or functional fragment thereof, is humanized or Humaneered™. In one aspect, the functional fragment is a Fab, F(ab)₂ Fv, or single chain Fv (scFv).

“Monoclonal antibody” means antibodies displaying a single binding specificity. “Monoclonal antibody” refers to an antibody that is the product of a single cell clone or hybridoma. Monoclonal antibodies can be prepared using a wide variety of methods known in the art including the use of hybridoma, recombinant, phage display and combinatorial antibody library methodologies, or a combination thereof. For example, monoclonal antibodies can be produced using hybridoma techniques including those known in the art and taught, for example, in Harlow and Lane, Antibodies: A Laboratory Manual, Cold Spring Harbor Laboratory Press (1989); Hammerling, et al., in: Monoclonal Antibodies and T-Cell Hybridomas 563-681, Elsevier, N.Y. (1981); Harlow et al., Using Antibodies: A Laboratory Manual, Cold Spring Harbor Laboratory Press (1999), and Antibody Engineering: A Practical Guide, C.A.K. Borrebaeck, Ed., W.H. Freeman and Co., Publishers, New York, pp. 103-120 (1991). Examples of known methods for producing monoclonal antibodies by recombinant, phage display and combinatorial antibody library methods, including libraries derived from immunized and naive animals can be found described in Antibody Engineering: A Practical Guide, C.A.K. Borrebaeck, Ed., supra. The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

As used herein, the term “functional fragment” when used in reference to the antibodies described herein is intended to refer to a portion of the antibody including heavy or light chain polypeptides which still retains some or all of the activity of the parent antibody molecule. Such functional fragments can include, for example, antibody functional fragments such as Fab, F(ab)₂ Fv, and single chain Fv (scFv). Other functional fragments can include, for example, heavy or light chain polypeptides, variable region polypeptides or CDR polypeptides or portions thereof so long as such functional fragments retain binding activity, specificity, inhibitory and activation activity. The term is also intended to include polypeptides encompassing, for example, modified forms of naturally occurring amino acids such as D-stereoisomers, non-naturally occurring amino acids, amino acid analogues and mimetics so long as such polypeptides retain functional activity as defined above.

A Fab fragment refers to a monovalent fragment consisting of the V_{L}, V_{H}, C_{L} and C_{H}1 domains; a F(ab′)₂ fragment is a bivalent fragment comprising two Fab fragments linked by a disulfide bridge at the hinge region; a Fd fragment consists of the V_{H} and C_{H}1 domains; an Fv fragment consists of the V_{L} and V_{H} domains of a single arm of an antibody; and a dAb fragment (Ward et al., Nature 341:544-546, (1989)) consists of a V_{H} domain.

An antibody can have one or more binding sites. If there is more than one binding site, the binding sites may be identical to one another or may be different. For example, a naturally occurring immunoglobulin has two identical binding sites, a single-chain antibody or Fab fragment has one binding site, while a “bispecific” or “bifunctional” antibody has two different binding sites.

A single-chain antibody (scFv) refers to an antibody in which a V_{L} and a V_{H} region are joined via a linker (e.g., a synthetic sequence of amino acid residues) to form a continuous polypeptide chain wherein the linker is long enough to allow the protein chain to fold back on itself and form a monovalent antigen binding site (see, e.g., Bird et al., Science 242:423-26 (1988) and Huston et al., Proc. Natl. Acad. Sci. USA 85:5879-83 (1988)). Diabodies refer to bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., Proc. Natl. Acad. Sci. USA 90:6444-48 (1993), and Poljak et al., Structure 2:1121-23 (1994)). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

“Binding specificity” of an antibody means the ability of an antibody to recognize an antigen to the exclusion of other antigens. This binding specificity is generally measured against nonspecific background binding or control and is typically considered specific when the antibody binds to the target antigen by at least 10 time above the background or control binding.

“Epitope” means a part of a molecule, for example, a portion of a polypeptide that specifically binds to one or more antibodies within the antigen binding site of the antibody. Epitopic determinants can include continuous or non-continuous regions of the molecule that binds to an antibody. Epitopic determinants also can include chemically active surface groupings of molecules such as amino acids or sugar side chains and have specific three dimensional structural characteristics and/or specific charge characteristics.

Antibody Humaneering™ generates engineered human antibodies with V-region sequences close to human germline sequence while retaining the specificity and affinity of a reference antibody as described in U.S. Patent Application Publications 2005-0255552 and 2006-0134098. The process identifies minimal sequence information, required to determine antigen-binding specificity from the Variable region of a reference antibody, and transfers that information to a library of human partial V-region gene sequences to generate an epitope focused library of human antibody V-regions. A microbial-based secretion system is used to express members of the library as antibody Fab fragments and the library is screened for antigen-binding Fabs using a colony-lift binding assay. Positive clones are further characterized to identify those with the highest affinity. The resultant engineered human Fabs retain the binding specificity of the parent, murine antibody, typically have equivalent or higher affinity for antigen than the parent antibody, and have V-regions with a high degree of sequence identity compared with human germ-line antibody genes.

“Bodily fluid” is any fluid derivable from the human body including fractions thereof. Lymph fluid, urine, mucus, whole blood, and amniotic fluid are examples of bodily fluids. Bodily fluids includes fractions of whole blood or other bodily fluids such as serum, plasma, cell free plasma and platelet free plasma.

“Comprising” means having at least the following but includes any and all additions (i.e. open claiming). Comprising necessarily encompasses “consisting essentially of” which is open to additions that do not change the fundamental nature or characteristics of the claimed subject matter. Both comprising and consisting essentially of necessarily encompass “consisting of” which means the expressly claimed subject matter without additions (i.e. closed claiming).

“Amplifying” means increasing the number DNA molecules having a specific sequence. The common means for amplifying DNA is PCR. However the term amplify encompasses any know technique in the art such as ligase chain reaction and cloning into high copy number plasmids.

“Enriching” means selectively increasing the relative proportion of one or more constituent in a heterogeneous mixture. Enrichment may encompass the loss of a portion of the enriched constituent relative to the total amount in a starting mixture. For example, fetal origin DNA from maternal plasma may be enriched relative to maternal DNA to produce a derivative mixture where the ratio of fetal:maternal DNA is increased, but some of the total fetal DNA in the maternal plasma is absent. Enrichment also encompasses increasing the relative proportion of a constituent in a part of a sample, for example, increasing the relative proportion of a constituent in a sample in the portion of the sample near a substrate surface.

“Fetal origin DNA” means DNA having originated from the genome of a fetus. Fetal origin DNA include for example DNA originating from trophoblastic tissues which are not part of the fetus per se but form the placenta or other tissues.

“Fetal Specific” means present in association with fetal origin materials in a heterogeneous mixture either exclusively or in relative greater proportion than the non-fetal origin constituents of the mixture. For example, a blood plasma sample with maternal and fetal microparticles may have a protein associated with both classes of microparticles, but more frequently or more accessibly on the fetal microparticles.

“Isolating” means separating a constituent from a starting material in any manner. For example, isolating microparticles in a volume of liquid may be done by ultracentrifugation to pellet the microparticles or immunoprecipitation from the volume of liquid.

“Ligand” means any molecule capable of specifically binding to a fetal specific protein or antigen present on a microparticle. Ligands include, for example, those that bind to Human Leukocyte Antigen-G (HLA-G), human placental alkaline phosphatase (hPLAP), integrin alpha-5 (CD49e), integrin alpha-v (CD51), integrin alpha-2 (CD49b), and integrin alpha-6 (CD49f).

Fetal Specific Proteins or Antigens

At least a portion of fetal origin microparticles in maternal bodily fluids are derived from apoptosis of trophoblastic cells, in particular extravillous trophoblasts and syncytiotrophoblasts. Fetal microparticles also arise from apoptosis of fetal cells, and placental cells. Other fetal microparticles are derived from nonapoptotic membrane particles that arise from fetal cells, trophoblast cells, and placental cells. Fetal microparticles contain proteins of fetal origin that are either fetal specific or at least disproportionately associated with fetal microparticles to permit relative enrichment from the other constituents of maternal bodily fluids. We refer generically to both types of proteins as “fetal specific proteins.” In one embodiment, fetal specific proteins do not include histones that meets the definition of a fetal specific protein. Differential fetal-maternal protein expression patterns and fetal tissue restricted protein expression are well documented. These “fetal specific proteins” constitute a large and well defined group of proteins. Each such protein will have one or more antigens to which antigen selective antibodies will bind.

While all fetal specific proteins or antigens are generally useful in the methods herein, a preferred subset of fetal specific proteins or antigens are those associated with the plasma membrane and having at least part of the protein as an extracellular domain. Further, the subset of fetal specific proteins or antigens expected to be associated with extravillous trophoblasts and syncytiotrophoblasts are particularly preferred. One member of both the plasma membrane subset and the trophoblast subset of proteins is Human Leukocyte Antigen-G (HLA-G). Another member of both these subsets is human placental alkaline phosphatase (hPLAP). Other exemplary fetal specific proteins or antigens include integrin alpha-5 (CD49e), integrin alpha-v (CD51), integrin alpha-2 (CD49b), integrin alpha-6 (CD49f), placental growth factor, NeuroD2, pregnancy-associated plasma protein-A (PAPP-A), and β-human chorionic gonadotrophin (FβhCG).

Antibodies to Fetal Specific Proteins or Antigens

Because the targeted fetal origin microparticles are apoptotic remnants, the fetal specific proteins and antigens associated therewith can exhibit varying degrees of degradation and denaturation. Consequently, we used several antibodies to both HLA-G and hPLAP. For HLA-G we screened the following commercially available antibodies:
Monoclonal antibody (mAb) 4H84 (IgG1), which recognizes the alpha-1 domain of HLA-G.
Anti-HLA-G mAb MEM-G/1 (IgG1), which also recognizes the alpha-1 domain.
mAb MEM-G/9 which recognizes the native form of HLA-G.
G233 (IgG2a) which recognizes the native form of HLA-G.
Biotinylated 87G (IgG2a) which recognizes the native form of HLA-G.
2A12 (IgG1) Mouse Monoclonal to HLA-G (soluble form).

Surprisingly, despite the apoptotic nature of the fetal microparticles, two of the six antibodies labeled fetal microparticles from maternal derived cell free plasma samples (2A12 and MEM-G/1). We also tested a single commercial antibody mAb H17E2 (IgG1) that binds hPLAP and determined that it was also effective for the methods herein. Thus, fetal specific proteins or antigens on microparticles can be targeted with antibodies for the methods of the invention.

Many other fetal specific proteins or antigens are suitable antibody targets for the production of antibodies suitable for the methods of the invention. For example, angiotensin II type 1 receptor (AT1), integrin alpha-5 (CD49e), integrin alpha-v (CD51), integrin alpha-2 (CD49b), and integrin alpha-6 (CD49f) are suitable fetal specific antigens for the methods of the invention. Preferred fetal specific antigens include HLA-G. PLAP, integrin alpha-5 (CD49e), integrin alpha-v (CD51), integrin alpha-2 (CD49b), and integrin alpha-6 (CD49f).

Ligands for Fetal Specific Proteins and Antigens

Ligands for fetal specific proteins, or fetal specific antigens on microparticles can also be used in the methods of the invention to enrich fetal microparticles from maternal samples. Ligands include, for example, those that bind to Human Leukocyte Antigen-G (HLA-G), human placental alkaline phosphatase (hPLAP), integrin alpha-5 (CD49e), integrin alpha-v (CD51), integrin alpha-2 (CD49b), and integrin alpha-6 (CD49f). Specific ligands include, for example, the fibronectin or the portion of fibronectin that binds to CD49e, or vitronectin or the portion of vitronectin that binds to CD51.

A ligand according to the present invention can be any compound, e.g., a peptide, polypeptide, nucleic acid, or small molecule. Preferred ligands include peptides, or polypeptides such as receptors for the polypeptide and fragments thereof comprising the binding domains for the peptides, and aptamers, e.g., nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides, or polypeptides. These derivatives can then be tested for binding according to screening procedures known in the art, e.g., phage display. Specific binding according to the present invention means that the ligand or agent should not bind substantially to (“crossreact” with) another peptide, polypeptide, or substance present in the sample to be analyzed. Preferably, the specifically bound polypeptide should be bound with at least 3 times higher, more preferably at least 10 times higher, and even more preferably at least 50 times higher affinity than any other relevant peptide or polypeptide. Non-specific binding may be tolerable, if it can still be distinguished and measured unequivocally, e.g., according to its size, or by its relatively higher abundance in the sample. Binding of the ligand can be measured by any method known in the art. Preferably, said method is semi-quantitative or quantitative.

The ligand can be attached to a substrate, such as, for example, a magnetic bead, the surface of microfluidic device, a matrix for column chromatography, and other substrates well-known in the art. The ligand may be coupled to the substrate with the use of a linker, such as, for example, a polymeric chain such as polyvinyl alcohol, or polyethylene glycol, and other molecules well-known in the art as linkers.

Fetal microparticles can be purified from a maternal sample by exposing the maternal sample to the ligand attached to a substrate. The fetal microparticle binds to the ligand and so becomes attached to the substrate allowing the fetal microparticle to be separated and enriched from the maternal sample.

Fetal Nucleic Acids

Fetal nucleic acids of the invention comprise any nucleic acid obtained from the microparticles enriched by the methods of the invention. These nucleic acids include, for example, DNA and/or RNA. The fetal nucleic acids of the invention are enriched at least five fold compared to the ratio of fetal to maternal nucleic acid in the maternal sample. Preferably, the fetal nucleic acids of the invention are enriched at least ten fold compared to the maternal sample. More preferably, the fetal nucleic acids are enriched twenty fold compared to the maternal sample. Still more preferably, the fetal nucleic acids of the invention are enriched twenty six fold compared to the maternal sample.

Maternal Bodily Fluids

Any maternal bodily fluid will serve as a source of fetal microparticles. Examples included lymphatic fluid, whole blood, plasma, serum, mucus, amniotic fluid, and urine. However, blood is the preferred maternal bodily fluid. While whole blood is a viable starting material, various fractions of blood can also be used such serum. Again, separation of blood for many routine clinical diagnostics is common practice and a preferred maternal bodily fluid is serum.

A preferred process was to take 5 to 10 ml of peripheral blood in vacutainer tubes containing 1.5 ml of ACD Solution A (trisodium citrate, 22.0 microliters; citric 114 acid, 8.0 microliters; and dextrose 24.5 microliters) no more than 24 hrs old. Plasma was separated from whole blood by centrifugation at 800×g for 10 minutes. Recovered plasma was centrifuged for an additional 10 minutes at 1,600×g to remove residual cells. Finally, cell-free supernatant was removed and stored in −80° C. freezer. This plasma separation method may be replaced with any other known in the art. For example, cell-free plasma samples are subjected to a further two-step centrifugation to obtain platelet free plasma (PFP). First, platelet poor plasma (PPP) is obtained by centrifugation speeds between 1,200-1,500×g for 10-20 minutes, followed by centrifugation speeds between 10,000-13,000×g for 30 minutes; the remaining supernatant contains Microparticles and is platelet free. To pellet apoptotic Microparticles/bodies, cell-free supernatants may be subjected a final centrifugation between 25,000-100,000×g. These pellets may then be resuspended in the medium and at the concentration of choice.

Other processes for separating whole blood are well-known in the art, for example, Separation of Human Blood and Bone Marrow Cells, (Ed. FMK Ali) John Wright, 1986 describes such methods, and is specifically incorporated by reference.

Screening for Antibodies or Ligands Using Microparticles

Starting with maternal blood plasma, a panel of anti-HLA-G and anti-PLAP antibodies was screened for effective reactivity with their target proteins on microparticles.

Prior to screening the antibodies the blood plasma sample was tested to measure the density of microparticles. Using this density, a standardized number of microparticles was used for each antibody screening test. A preferred method of microparticle quantification is described in Martin Montes, Elin A. Jaensson, Aaron F. Orozco, Dorothy E. Lewis, David B. Corry, A general method for bead-enhanced quantitation by flow cytometry, Journal of Immunological Methods, Volume 317, Issues 1-2, 20 Dec. 2006, Pages 45-55, ISSN 0022-1759, DOI: 10.1016/j.jim.2006.09.013, which is specifically incorporated herein by reference. Briefly, fluorescent beads (20,000) were added to a 1:53.3 dilution of (15 microliters) plasma in double filtered (0.25 micrometer) PBS (dfPBS) for a total volume of 800 microliters and a final concentration of 25 beads/microliter. The number of beads counted by an EPICS XL-2 flow cytometer (Beckman Coulter) was stopped at 1,000. Other processes well known in the art may be used to measure the density of microparticles. E.g., Ibid. at Table 1.

A similar approach may be taken for the screening of ligands that may be used in the methods of the invention.

Screening for Antibodies or Ligands Using Cell Lines

Fetal microparticles can also be approximated by using by using certain cell lines.

For example HTR-8/SVneo is an available trophoblastic cell line and JEG-3 cells are an available extravillous cytotrophoblast cell line. Cell cultures of these cell lines may be induced to undergo apoptosis and release apoptotic microparticles using known methods. Orozco A F, Jorgez C J, Horne C, Marquez-Do D A, Chapman M R, Rodgers J R, Bischoff F Z, Lewis D E. Membrane protected apoptotic trophoblast microparticles contain nucleic acids: relevance to preeclampsia. Am J Pathol 2008; 173:1595-608, hereby specifically incorporated herein by reference. These cell culture based models of fetal origin microparticle formation can be used as a first screen for antibodies or ligands that will interact with fetal specific proteins associated with fetal microparticles. For example, the antibody or ligand may be conjugated to a fluorescent label, the labeled antibody or ligand is exposed to the trophoblast cell line, and the cells are monitored for associated fluorescence.

Candidate antibodies and ligands that interact with the cell line can then be screened against maternally derived fetal microparticles to identify those antibodies and ligands suitable for the methods of the invention.

Using Fetal Specific Proteins to Isolate Microparticles with Fetal DNA

Having demonstrated the ability to immunologically label microparticles containing fetal DNA, we next examined using antibodies bound to fetal specific proteins associated with microparticles as a tool for enriching and purifying fetal origin microparticles from the background maternal microparticles. Immunopurification procedures such as immunoaffinity chromatography and immunoprecipitations are diverse and well known. Timothy A. Springer, 2001. Immunoaffinity Chromatography. Curr. Protoc. Protein Sci. Unit 9.5; Juan S. Bonifacino, et al., 2001. Immunoprecipitation. Curr. Protoc. Protein Sci. Unit 9.8. Immunofluorescence based sorting by flow cytometry is another immunopurification procedure, hereby specifically incorporated by reference. To test the adaptability for isolating [{microparticle+fetal DNA}−fetal specific protein-antibody] complexes by immunopurification, we selected immunoprecipitation as a representative immunopurification technique. One reason for choosing immunoprecipitation is the low cost, speed and simplicity of this immunopurification technique relative to e.g. chromatographic and flow cytometry techniques.

Microparticles can also be enriched using affinity chromatography by attaching the antibody or ligand specific for the fetal antigen or protein to a column support. Fetal microparticles can then be enriched by passing the maternal sample through the affinity column that will preferentially bind to the fetal microparticles.

The antibodies or ligands may also be coupled to the substrate surface of a microfluidic device, instead of column support material. Fetal microparticles are then enriched by passing the maternal sample through the microfluidic device.

In a preferred embodiment, antibodies or ligands for fetal antigens or proteins are coupled to magnetic beads or particles, and fetal microparticles are enriched using standard isolation techniques based on magnetic particles. For example, Stemcell Technologies sells a product called EasySep® that uses antibodies specific to a target combined with anti-dextran antibody fragments, and dextran coated magnetic beads. The target specific antibody and anti-dextran antibody are coupled together and link the target to the magnetic bead. Target is then isolated using the magnetic properties of the bead (or nanoparticle).

Using Fetal DNA from Microparticles for Prenatal Diagnostics

Because paternally inherited genetic material will be amplified sufficiently to be detected, the single immunopurification enrichment protocol described herein will for the first time make routine prenatal genetic analysis feasible. The DNA from microparticles from maternal bodily fluids is suitable for most genetic testing (other than FISH or other chromosomal analysis). For example, enriched microparticle DNA may be used to PCR amplify a sequence associated with a SNP followed by sequencing to detect a doublet signal at the polymorphic position by fluorescent tagged sequencing. Of course, it is contemplated that further purifications could be applied in series such as flow cytometry separation or immunoprecipitation by a second antibody to a different fetal specific protein prior to DNA extraction and analysis. A nonexhaustive list of commonly used genetic testing follows:

Gender (sex determination)

Aneuploidy

RhD type (rhesus D status determination)

2,4-Dienoyl-CoA reductase deficiency

2-Methylbutryl-CoA dehydrogenase deficiency

3-Methylcrotonyl-CoA carboxylase deficiency (3MCC)

3-Methylglutaconyl-CoA hydratase deficiency

3-OH 3-CH3 glutaric aciduria; 3-hydroxy-3-methylglutaryl-CoA lyase deficiency

5-Oxoprolinuria (pyroglutamic aciduria)

Adrenal hyperplasia

Argininemia

Argininosuccinic acidemia (ASA)

Beta-ketothiolase deficiency (BKT)

Biotimidase deficiency (BLOT)

Carbamoylphosphate synthetase deficiency (CPS def.)

Carnitine uptake defect (CUD)

Citrullinemia (CITR)

Congenital adrenal hyperplasia (CAH)

Congenital hypothyroidism

Cystic fibrosis (CF)

Down's syndrome

Fragile-X syndrome

Galactosemia

Glucose-6-Phosphate dehydrogenase deficiency (G6PD)

Glutaric acidemia type I (GA-I)

Hemoglobinopathy

Hexosaminidase A (Tay-Sachs disease)

Homocystinuria

Hyperammonemia, hyperornithinemia, homocitrullinemia syndrome (HHH)

Hyperornithine with gyrate deficiency

Isobutyryl-CoA dehydrogenase deficiency

Isovaleric acidemia (IVA)

Long-chain L-3-OH acyl-CoA dehydrogenase deficiency (LCHADD)

Malonic aciduria

Maple syrup urine disease (MSUD)

Medium chain acyl-CoA dehydrogenase deficiency (MCADD)

Methylmalonic acidemia

Multiple acyl-CoA dehydrogenase deficiency (MADD)

Multiple carboxylase deficiency (MCD)

Neonatal carnitine palmitoyl transferase deficiency-type II (CPT-II)

Phenylketonuria (PKU)

Propionic acidemia (PROP)

Short chain acyl-CoA dehydrogenase deficiency (SCAD)

Short chain hydroxy acyl-CoA dehydrogenase deficiency (SCHAD)

Tay-Sachs disease

Trifunctional protein deficiency (TFP)

Tyrosinemia type I (TYRO-I)

Very long-chain acyl-CoA dehydrogenase deficiency (VLCAD)

Duchenne muscular dystrophy

Thalassemia

Sickle cell anemia

Congenital adrenal hyperplasia

Huntington disease

Type 1 diabetes

BRCA 1 and 2

Any genetic test based on analysis of single nucleotide polymorphisms (SNPs), microsatellite sequences or restriction fragment length polymorphisms.

The enriched fetal nucleic acids obtained by the methods of the invention may be tested for any of the above, or for other well-known prenatal diagnostics using techniques well-known in the art including, for example, polymerase chain reaction (PCR), real-time polymerase chain reaction (RT-PCR), ligase chain reaction (LCR), self-sustained sequence replication (3SR) also known as nucleic acid sequence based amplification (NASBA), Q-B-Replicase amplification, rolling circle amplification (RCA), transcription mediated amplification (TMA), linker-aided DNA amplification (LADA), multiple displacement amplification (MDA), invader and strand displacement amplification (SDA), digital PCR (dPCR), or combinations of any of these.

The enriched fetal nucleic acids obtained by the methods of the present invention can be used to conduct genetic tests or screening of a fetus. In particular, the enriched nucleic acids can be used to test or screen the genetic composition of a fetus, e.g. chromosomal composition, gene composition, or genetic marker or finger printing pattern of a fetus. In one embodiment, testing or screening a genetic composition of a fetus includes probing for chromosomal abnormalities including, without any limitation, monosomy, partial monosomy, trisomy, partial trisomy, chromosomal translocation, chromosomal duplication, chromosomal deletion or microdeletion, and chromosomal inversion.

In general, the term “monosomy” refers to the presence of only one chromosome from a pair of chromosomes. Monosomy is a type of aneuploidy. Partial monosomy occurs when the long or short arm of a chromosome is missing. Common human genetic disorders arising from monosomy include: X0, only one X chromosome instead of the usual two (XX) seen in a normal female (also known as Turner syndrome); cri du chat syndrome, a partial monosomy caused by a deletion of the end of the short p (from the word petit, French for small) arm of chromosome 5; and 1p36 Deletion Syndrome, a partial monosomy caused by a deletion at the end of the short p arm of chromosome 1.

In contrast, the term “trisomy” refers to the presence of three, instead of the normal two, chromosomes of a particular numbered type in an organism. Thus the presence of an extra chromosome 21 is called trisomy 21. Most trisomies, like most other abnormalities in chromosome number, result in distinctive birth defects. Many trisomies result in miscarriage or death at an early age. A partial trisomy occurs when part of an extra chromosome is attached to one of the other chromosomes, or if one of the chromosomes has two copies of part of its chromosome. A mosaic trisomy is a condition where extra chromosomal material exists in only some of the organism's cells. While a trisomy can occur with any chromosome, few babies survive to birth with most trisomies. The most common types that survive without spontaneous abortion in humans include: Trisomy 21 (Down syndrome); Trisomy 18 (Edwards syndrome); Trisomy 13 (Patau syndrome); Trisomy 9; Trisomy 8 (Warkany syndrome 2); Trisomy 16 (which is the most common trisomy in humans, occurring in more than 1% of pregnancies. This condition, however, usually results in spontaneous miscarriage in the first trimester). Trisomy involving sex chromosomes include: XXX (Triple X syndrome); XXY (Klinefelter's syndrome); and XYY (XYY syndrome).

In another embodiment, testing or screening a genetic composition of a fetus includes probing for allele or gene abnormalities, e.g., one or more mutations such as point mutations, insertions, deletions in one or more genes.

In yet another embodiment, testing or screening a genetic composition of a fetus includes probing for one or more polymorphism patterns or genetic markers, e.g., short tandem repeat sequences (STRs), single nucleotide polymorphisms (SNPs), etc.

In still another embodiment, testing or screening a genetic composition of a fetus includes probing for any genetic abnormality corresponding to or associated with a condition or disorder, e.g. Cystic Fibrosis, Sickle-Cell Anemia, Phenylketonuria, Tay-Sachs Disease, Adrenal Hyperplasia, Fanconi Anemia, Spinal Muscularatrophy, Duchenne's Muscular Dystrophy, Huntington's Disease, Beta Thalassaemia, Myotonic Dystrophy, Fragile-X Syndrome, Down Syndrome, Edwards Syndrome, Patau Syndrome, Klinefelter's Syndrome, Triple X syndrome, XYY syndrome, Trisomy 8, Trisomy 16, Turner Syndrome, Robertsonian translocation, Angelman syndrome, DiGeorge Syndrome, Wolf-Hirschhom Syndrome, RhD Syndrome, Tuberous Sclerosis, Ataxia Telangieltasia, and Prader-Willi syndrome.

In still another embodiment, testing or screening a genetic composition of a fetus includes probing for any genetic condition corresponding to or associated with gender or paternity of the fetus.

In a particular embodiment, genetic tests provided by the present invention use the nucleic acids obtained by the methods of the present invention either directly or as templates for “amplification-based” genetic composition testing assays, including without any limitation, PCR, RT-PCR, LCR, 3SR, NASBA, RCA, TMA, LADA, MDA, SDA and dPCR. Amplification of a nucleotide fragment using a pair of primers specific for an allele indicates the presence of the allele.

In one embodiment, the “amplification-based” genetic composition testing assays of the present invention include using primers to generate amplicons less than about 200 base pairs, less than about 150 base pairs, or between about 75 to about 150 base pairs.

In a particular embodiment, the enriched fetal nucleic acids obtained by the methods of the present invention can be used to conduct genetic tests or screening to identify a chromosome aneuploidy in a chromosome of a fetal cell. “Chromosome aneuploidy in a chromosome” as used herein includes a chromosome missing or having an extra copy or part of a chromosome as compared to the normal native karyotype of a subject and includes deletion, addition and translocation, which causes monosomy or trisomy at particular sites. Preferably the aneuploidy is selected from the group including monosomy and trisomy of autosomes, and monosomy, disomy and trisomy of sex chromosomes.

### EXAMPLES

### HLA-G Antibody MEM-G/1 and hPLAP Antibody H17E2

One million microparticles in 66 microliters dfPBS were labeled with 3 micrograms of MEM-G/1 or isotype control and mixed on a BD ADAMS Nutator (Aria Medical Equipment) for 6 min. In a separate reaction, microparticles were labeled with 1 microgram hPLAP or isotype control mAb and mixed as before. The antibody:microparticles conjugates were then labeled with fluorescent secondary antibody (Phycoerythrin-goat antimouse immunoglobulin G, 0.8 microgram) and mixed 5 min. Afterwards, microparticles were labeled with a double stranded DNA stain (2 microliters of PicoGreen®) for 10 min in the dark. Double stranded DNA staining was performed so that we could assess the number of microparticles actually associated with measurable amounts of DNA. Unlabeled Microparticles were also used as negative controls. Both labeled and unlabeled Microparticles were re-suspended in 133 microliters dfPBS and analyzed by an EPICS XL-2 flow cytometer. The number of events was stopped at 10,000 counts.

HLA-G Antibody MEM-G/1

Plasma samples tested for HLA-G antibody MEM-G/1 corresponded to 31 pregnant women between 7 and 36 weeks gestation and 11 non-pregnant controls, 6 females and 5 males. MP levels are summarized in Table 1.

TABLE 1
Most HLA-G⁺ mpDNA detected in first trimester

| Non-pregnant | Maternal | First | Second | Third |
|---|---|---|---|---|
| controls | Plasma | Trimester | Trimester | Trimester |
| (n = 11) | (n = 31) | (n = 13) | (n = 15) | (n = 3) |
| 1.5 ± 1.2 | *21.2 ± 21.4 | *33.1 ± 28.0 | *14.5 ± 7.0 | 3.2 ± 2.0 |

Data are reported as mean ± SD and compared to non-pregnant controls using one-way ANOVA
*Differs significantly from control group (p < 0.0005)
HLA-G (Human leukocyte antigen-G)
mpDNA (Micro-particles containing DNA)

The mean percentage of HLA-G+/DNA+ microparticles was fourteen-fold higher in maternal plasma (21.2%, n=31) compared to plasma from non-pregnant controls (1.5%, n=11; P=0.0001) (Table 1). HLA-G+/DNA+ microparticles were detected at all stages of gestation with the highest amount found in first trimester (33.1%, n=13; P=0.0001), followed by second trimester (14.5%, n=15; p=0.0001) and then third trimester (3.3%, n=3; P=0.5) (Table 1).

hPLAP Antibody H17E2

Additional frozen aliquots of the same plasma samples used for HLA-G labeling experiments were used to detect hPLAP+ microparticles. Fifteen maternal plasma samples ranging between 9 and 36 weeks of gestation and 8 non-pregnant controls (5 females and 3 males) were tested. The results are summarized in Table 2:

TABLE 2
Most PLAP⁺ mpDNA detected in second trimester

| Non-pregnant | Maternal | First | Second | Third |
|---|---|---|---|---|
| controls | Plasma | Trimester | Trimester | Trimester |
| (n = 8) | (n = 15) | (n = 6) | (n = 6) | (n = 3) |
| 0.9 ± 0.6 | ***9.8 ± 7.4 | **9.1 ± 7.2 | ***12.3 ± 9.2 | *6.4 ± 0.7 |

Data are reported as mean ± SD and compared to non-pregnant controls using one-way ANOVA
*Differs significantly from control group (p < 0.05)
**Differs significantly from control group (p < 0.005)
***Differs significantly from control group (p < 0.0005)
PLAP (Placental alkaline phosphatase)
mpDNA (Micro-particles containing DNA)

The mean percentage of hPLAP+/DNA+ microparticles was eleven-fold higher in maternal plasma samples (9.8%, n=15) compared to plasma from non-pregnant controls (0.9%, n=8; P=0.001) (Table 2). Compared to hPLAP+/DNA+ microparticles levels during first trimester (9.1%, n=6), amounts of hPLAP+/DNA+ microparticles increased during second trimester (12.3%, n=6, p=0.0004) and declined by the third trimester (6.1%, n=3, P=0.01) (Table 2).

Immunoprecipitation with Magnetic Beads

We tested the MEM-G/1 mouse mAb against HLA-G for microparticle immunoprecipitation. Any immunoprecipitation protocol will be sufficient. However throughout our process design, we chose commonly used laboratory techniques with commercially available reagents. We chose one of the many commercially available kits for immunoprecipitation, EasySep “Do-it-yourself” (StemCell Technologies). Briefly, 30 micrograms of MEM-G/1 was added to a 1.5 ml polypropylene tube. 100 microliters of component A (mouse mAb against dextran) was added to the tube and vortexed. 100 microliters of component B (mAb against the Fc region of mouse IgG) was added to the tube and vortexed. The tube was wrapped in PARAFILM “M” (Pechiney Plastic Packaging) and placed in a 37° C. water bath overnight (12 hrs) to form a tetrameric antibody complex (MEM-G/1+ anti-dextran mAbs). The next day, the tetrameric antibody complex was brought to a final volume of 1.0 ml with dfPBS. All isolation procedures were done at room temperature. 50 microliters of the cocktail was added per 800 microliters of plasma and mixed as before for 20 min. 25 microliters of dextran-coated magnetic nanoparticles were added to the sample and mixed for 10 min. The sample was allowed to settle for an additional 10 min, transferred to a 5.0 ml polystyrene round-bottom tube (BD Bioscience) and the volume was brought up to 2.5 ml with dfPBS+1 mM EDTA. The tube was placed on a magnet (StemCell Technologies) for 10 min. The magnet and the tube were inverted in one continuous motion, pouring off the supernatant. The magnet was returned to an upright position and the residual fluid allowed to settle for 5 min.

We applied the above protocol to 11 plasma samples from pregnant women (13-35 weeks of gestation) carrying a male fetus. Plasma from women carrying a female fetus (n=3) was used as negative controls. DNA associated with the immunoprecipitated microparticles was directly purified from the nanoparticle beads using a commercially available reagent for processing blood samples (QIAamp DNA blood kit (Qiagen)).

Anti-AT1 Antibodies

We chose another exemplary fetal specific protein, angiotensin II type 1 receptor (AT1). AT1 is fetal specific in the sense that it is expressed by trophoblastic tissues and we expected that AT1 would be quantitatively more associated with fetal origin microparticles than maternal origin microparticles in blood plasma.

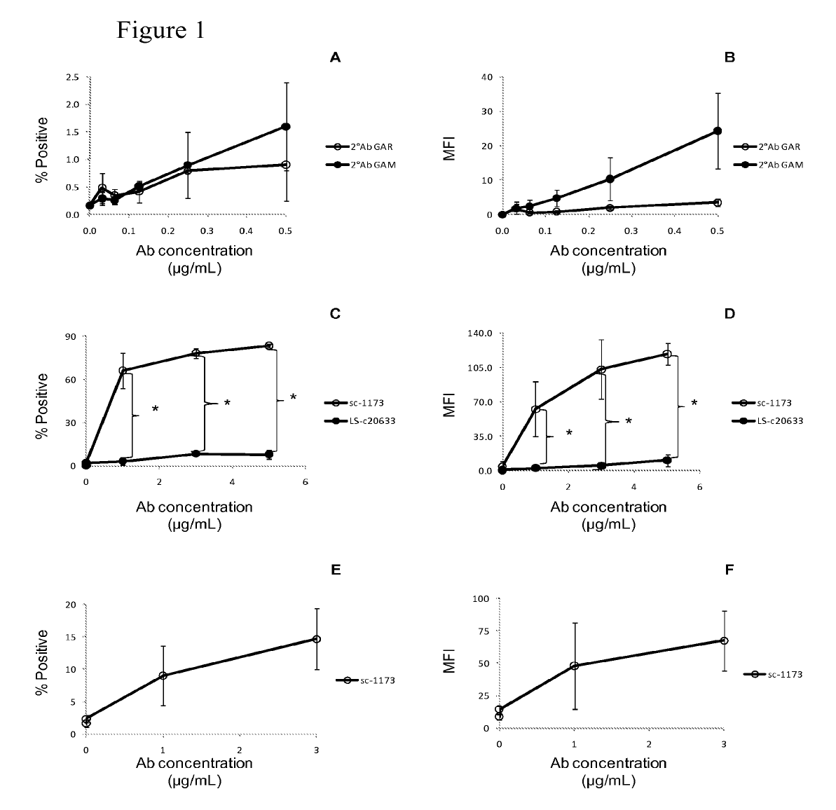

One million microparticles were re-suspended in dfPBS and labeled with two commercially available anti-AT1 Abs and mixed on a BD ADAMS Nutator (Aria Medical Equipment) for 15 min. MPs were then labeled with secondary PE-conjugated GAR and mixed for another 15 min. Afterwards, MPs were re-suspended in a total volume of 500 microliters dfPBS and analyzed by an LSR II flow cytometer. The number of events was stopped at 10,000 counts. Data collected from the experiments were analyzed using Summit V3.1 (analysis software from DAKO). The results are shown in FIG. 1.

Our results with AT1 again confirm that fetal specific proteins as a group are useful targets for the methods herein and effective antibodies thereto may be readily identified. Further, induced apoptotic microparticles from cell lines are shown to be an effective alternative to maternal bodily fluid for screening for fetal specific proteins and antibodies thereto. Cell culture derived microparticles further allow for optimization and optimal selection of antibodies for microparticle labeling without the need for medical samples of bodily fluids.

Additional fetal specific antigens were also tested. Invasive extravillous cytotrophoblast express surface markers such as HLA-G, integrin alpha-5 (CD49e), and integrin alpha-v (CD51), whereas proliferating extravillous cytotrophoblast HLA-G, integrin alpha-2 (CD49b), and integrin alpha-6 (CD490. We used our established in vitro apoptotic MP system to optimally label apoptotic MPs derived from JEG-3 cells (extravillous cytotrophoblast cell line), which express integrin alpha-5 (CD49e) and integrin alpha-v (CD51). We applied polychromatic fluorescent cytometry using CD49e-FITC, CD51-FITC purchased from BioLegend (San Diego, Calif.). We confirmed the ability of these fetal specific proteins to label microparticles (data not shown). Flow cytometry sorting will further allow for a highly enriched source of fetal origin microparticles with DNA based on DNA staining and multiple antibody labeling.

Analysis of DNA Associated with Microparticles

We analyzed DNA extracted from the immunoprecipitated microparticles by Real-time PCR as previously described. Jorgez C J, Dang D O, Simpson J L, Lewis D E, Bischoff F Z. Quantity versus quality: optimal methods for cell-free DNA isolation from plasma of pregnant women. Genet Med 2006; 8:615-9, hereby specifically incorporated herein by reference. For both the beta-globin (102 bp) and Sex-determining Region Y (SRY) (72 bp), quantitative real-time PCR was performed using the Applied Biosystems 7700 sequence detection system (Applied Biosystems). Primer and probe sequences were as follows:

SRY forward primer:
5′-TGC ACA GAG AGA AAT ACC CGAAn A-3′
SRY reverse primer:
5′-TGC An CTT CGG CAG CAT-3′:
SRY TaqMan probe:
5′-AAG TAT CGA CCT CGT CGG AAG GCG AA-3′
Beta-globin forward primer:
5′-GTG CAC CTG ACT CCT GAG GAG A-3′
Beta-globin reverse primer:
5′-CCTTGA TAC CAA CCT GCC CAG-3′
Beta-globin TaqMan probe:
5′-AAG GTG AAC GTG GAT GAA GTT GGT GG-3′

Quantification of total and fetal DNA as genome equivalents was based on copies of Beta-globin and SRY sequences. Each reaction contained 5 microliters of DNA extracted from immunoprecipitated microparticles. Each reaction plate was run simultaneously with duplicate calibration curves of titrated DNA (standard curve). Each sample was run in duplicate for both loci and the mean of the values was determined using the 7700 software and the standard curve of known DNA concentrations. Quantification of fetal DNA enrichment was determined by the ratio of SRY to beta-globin before and after immunoprecipitation with magnetic beads. The results are shown in Table 3. Two (13.3 and 15.4 weeks) of the eleven samples had low amounts of total DNA (Table 3) and were therefore excluded from the statistical calculations.

TABLE 3
Quantification of fetal and total cfDNA in maternal plasma before and
after enrichment of HLA-G⁺ MPs.

| Gestational Age | | Plasma | SRY | b-glo | | Fold-Enrichment |
|---|---|---|---|---|---|---|
| (weeks) | Fetus | Treatment | (Geq/ml) | (Geq/ml) | % Fetal DNA | of Fetal DNA |
| 13.1 | XY | Non-Enriched | 42.5 | 1100.0 | 3.9 | 22.4 |
| | | Enriched | 11.8 | 13.6 | 86.6 | |
| 13.3¹ | XY | Non-Enriched | 51.8 | 862.0 | 6.0 | 0.7 |
| | | Enriched | 8.3 | 189.4 | 4.4 | |
| 14.6 | XY | Non-Enriched | 72.1 | 1256.4 | 5.7 | 5.2 |
| | | Enriched | 40.9 | 136.5 | 30.0 | |
| 15.4² | XY | Non-Enriched | 125.7 | 1119.3 | 11.2 | 0.7 |
| | | Enriched | 12.5 | 169.9 | 7.4 | |
| 18.9 | XY | Non-Enriched | 17.3 | 4429.5 | 0.4 | 14.8 |
| | | Enriched | 12.8 | 220.5 | 5.8 | |
| 19.4 | XY | Non-Enriched | 39.0 | 2163.5 | 1.8 | 8.2 |
| | | Enriched | 16.3 | 110.5 | 14.7 | |
| 20.0 | XY | Non-Enriched | 71.7 | 3150.0 | 2.3 | 35.7 |
| | | Enriched | 135.8 | 167.1 | 81.2 | |
| 22.0 | XY | Non-Enriched | 67.3 | 1988.3 | 3.4 | 8.0 |
| | | Enriched | 45.5 | 167.5 | 27.2 | |
| 23.0 | XY | Non-Enriched | 20.0 | 3517.0 | 0.6 | 127.6 |
| | | Enriched | 74.8 | 103.0 | 72.6 | |
| 26.6 | XY | Non-Enriched | 22.4 | 1284.5 | 1.7 | 7.2 |
| | | Enriched | 28.5 | 228.4 | 12.5 | |
| 35.0 | XY | Non-Enriched | 37.0 | 872.0 | 4.2 | 3.1 |
| | | Enriched | 25.3 | 191.5 | 13.2 | |
| 12.7 | XX | Non-Enriched | 0.0 | 2730.0 | No signal | No signal |
| | | Enriched | 0.0 | 95.8 | | |
| 14.0 | XX | Non-Enriched | 0.0 | 2810.6 | No signal | No signal |
| | | Enriched | 0.0 | 346.0 | | |
| 20.0 | XX | Non-Enriched | 0.0 | 3324.1 | No signal | No signal |
| | | Enriched | 0.0 | 277.9 | | |

¹Omitted from statistical calculations
²Omitted from statistical calculations
cfDNA (Cell-free DNA)
MPs (Micro-particles)
SRY (Sex determining region Y)
b-glo (β-globin)

Prior to enrichment of fetal DNA, 2195.7±1242.8 Geq/ml beta-globin and 43.2±22.2 Geq/ml SRY were detected, whereas 148.7±67.1 Geq/ml beta-globin and 43.5±40.0 Geq/ml SRY were detected after enrichment. Together these data suggest that immunoprecipitated of microparticles from maternal plasma using antibodies to fetal specific proteins can greatly increase the percentage of fetal DNA (38.2±32.5%) relative to the total cell free DNA in maternal plasma (2.7±1.8%, n=9, p=0.0003). SRY was not detected in non-enriched or enriched plasma samples from women carrying a female fetus (n=3). Overall, these data show that in 9 of 9 samples, a average 26-fold enrichment in fetal DNA was achieved using a magnetic bead technique that is far less costly and less complicated than previously available enrichment procedures and thus feasible in routine clinical settings.

Most genetic testing involves amplification and detection comparable to that used for beta-globin and SRY. However, we further assessed the quality of DNA associated with apoptotic microparticles produced using our cell culture systems. DNA fragmentation was tested by the terminal deoxynucleotidyl transferase dUTP nick end labeling (TUNEL) assay. Microparticles were labeled with Bromodeoxyuridine triphosphate (Br-dUTP), counterstained with propidium iodide, and analyzed by flow cytometry. Gel electrophoresis showed that DNA from these apoptotic microparticles displayed a disrupted DNA ladder pattern in roughly 180 by increments, similar to apoptotic cellular DNA. While fragmented, this apoptotic DNA will be suitable for use as a substrate for, e.g., PCR amplification or even direct cycle sequencing. The successful amplification of the segment of the SRY in our samples (Table 3) proves that paternally inherited alleles will be efficiently amplified from the enriched DNA.

Comparison to Preeclampsia Samples

Certain medical conditions result in elevated maternal microparticle content in maternal blood. González-Quintero V H, Smarkusky L P, Jimenez J J, Mauro L M, Jy W, Hortsman L L, O'Sullivan M J, Ahn Y S., Elevated plasma endothelial microparticles: preeclampsia versus gestational hypertension. Am J Obstet Gynecol. 2004 October; 191(4):1418-24. We obtained samples representing Preeclampsia to test the robustness and specificity of antibody labeling for fetal specific proteins.

The concentration of total microparticle (maternal and fetal) in term preeclamptic and control plasma samples was analyzed using the same bead counting flow cytometric method described above. The results are shown in Table 3:

TABLE 3
Plasma from preeclamptic women has more MPs than
plasma from normal pregnancies.

| | Control | Preeclamptic |
|---|---|---|
| | Pregnancy | Pregnancy |
| | (n = 9) | (n = 11) |
| MPs | 7.9 ± 3.7 | *14.1 ± 6.6 |
| mpDNA | 2.9 ± 1.7 | **6.1 ± 4.3 |
| HLA-G⁺ mpDNA | 0.3 ± 0.4 | **1.1 ± 1.4 |
| PLAP⁺ mpDNA | 0.9 ± 0.9 | 0.4 ± 0.3 |

Data are reported as mean ± SD
MP concentration (MPs/ml) × 10⁷
Statistical analysis used: two-sample t-test after natural log transformation
*Differs significantly from control pregnancy (p < 0.05)
**Different from control pregnancy (p = 0.07)
MPs (Micro-particles)
mpDNA (Micro-particles containing DNA)
HLA-G (Human leukocyte antigen-G)
PLAP (Placental alkaline phosphatase)

As expected, total microparticles in preeclamptic plasma (14.1±6.6×10⁷ MPs/ml, n=11) were significantly higher compared to control plasma as determined by Student's t-test (7.9±3.7×10⁷ MPs/ml, n=9, P=0.03). In contrast, the concentration of total (maternal and fetal) DNA+ microparticles in preeclamptic plasma samples (6.1±4.2×10⁷ MPs/ml, n=11) was only minimally higher compared to plasma from control pregnancies (2.9±1.7×10⁷ MPs/ml, n=9, P=0.07) (Table 3). HLA-G+/DNA+ microparticles in preeclamptic plasma samples (1.1±1.4×10⁷ MPs/ml, n=11) were minimally higher compared to control plasma samples (0.3±0.4×10⁷ MPs/ml, n=9, P=0.07) (Table 3). This result demonstrates that an increased background of maternal microparticles does not interfere with our ability to discern DNA+, HLA-G+/DNA+, or hPLAP+/DNA+ microparticles.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

The following references and any cited in the preceding Disclosure are hereby incorporated by reference in their entireties and in particular for any content for which a reference is specifically cited.
1. Thery C, Ostrowski M, Segura E. Membrane vesicles as conveyors of immune responses. Nat Rev Immunol 2009; 9:581-93.
2. Valadi H, Ekstrom K, Bossios A, Sjostrand M, Lee J J, Lotvall J O. Exosome-mediated transfer of mRNAs and microRNAs is a novel mechanism of genetic exchange between cells. Nat Cell Biol 2007; 9:654-9.
3. Luo S S, Ishibashi O, Ishikawa G, Ishikawa T, Katayama A, Mishima T, Takizawa T, Shigihara T, Goto T, Izumi A and others. Human Villous Trophoblasts Express and Secrete Placenta-Specific MicroRNAs into Maternal Circulation via Exosomes. Biol Reprod 2009.
4. Thery C, Boussac M, Veron P, Ricciardi-Castagnoli P, Raposo G, Garin J, Amigorena S. Proteomic analysis of dendritic cell-derived exosomes: a secreted subcellular compartment distinct from apoptotic vesicles. J Immunol 2001; 166:7309-18.
5. Arteaga R B, Chirinos J A, Soriano A O, Jy W, Horstman L, Jimenez J J, Mendez A, Ferreira A, de Marchena E, Ahn Y S. Endothelial microparticles and platelet and leukocyte activation in patients with the metabolic syndrome. Am J Cardiol 2006; 98:70-4.
6. Miksa M, Wu R, Dong W, Komura H, Amin D, Ji Y, Wang Z, Wang H, Ravikumar T S, Tracey K J and others. Immature Dendritic Cell-Derived Exosomes Rescue Septic Animals Via Milk Fat Globule Epidermal Growth Factor VIII. J Immunol 2009.
7. Saunderson S C, Schuberth P C, Dunn A C, Miller L, Hock B D, MacKay P A, Koch N, Jack R W, McLellan A D. Induction of exosome release in primary B cells stimulated via CD40 and the IL-4 receptor. J Immunol 2008; 180:8146-52.
8. Wang G J, Liu Y, Qin A, Shah S V, Deng Z B, Xiang X, Cheng Z, Liu C, Wang J, Zhang L and others. Thymus exosomes-like particles induce regulatory T cells. J Immunol 2008; 181:5242-8.
9. Sellam J, Proulle V, Jungel A, Ittah M, Miceli-Richard C, Gottenberg J E, Toti F, Benessiano J, Gay S, Freyssinet J M and others. Increased levels of circulating microparticles in primary Sjogren's syndrome, systemic lupus erythematosus and rheumatoid arthritis, and relation with disease activity. Arthritis Res Ther 2009; 11:R156.
10. Orozco A F, Jorgez C J, Ramos-Perez W D, Popek E J, Yu X, Kozinetz C A, Bischoff F Z, Lewis D E. Placental Release of Distinct DNA-associated Microparticles into Maternal Circulation: Reflective of Gestation Time and Preeclampsia. Placenta 2009.
11. Mitchell P J, Welton J, Staffurth J, Court J, Mason M D, Tabi Z, Clayton A. Can urinary exosomes act as treatment response markers in prostate cancer? J Transl Med 2009; 7:4.
12. Admyre C, Johansson S M, Qazi K R, Filen J J, Lahesmaa R, Norman M, Neve E P, Scheynius A, Gabrielsson S. Exosomes with immune modulatory features are present in human breast milk. J Immunol 2007; 179:1969-78.
13. Houali K, Wang X, Shimizu Y, Djennaoui D, Nicholls J, Fiorini S, Bouguermouh A, Ooka T. A new diagnostic marker for secreted Epstein-Barr virus encoded LMP1 and BARF1 oncoproteins in the serum and saliva of patients with nasopharyngeal carcinoma. Clin Cancer Res 2007; 13:4993-5000.
14. Hawari F I, Rouhani F N, Cui X, Yu Z X, Buckley C, Kaler M, Levine S J. Release of full-length 55-kDa TNF receptor 1 in exosome-like vesicles: a mechanism for generation of soluble cytokine receptors. Proc Natl Acad Sci USA 2004; 101:1297-302.
15. Williams J B, Jauch E C, Lindsell C J, Campos B. Endothelial microparticle levels are similar in acute ischemic stroke and stroke mimics due to activation and not apoptosis/necrosis. Acad Emerg Med 2007; 14:685-90.
16. Nusbaum P, Laine C, Seveau S, Lesavre P, Halbwachs-Mecarelli L. Early membrane events in polymorphonuclear cell (PMN) apoptosis: membrane blebbing and vesicle release, CD43 and CD16 down-regulation and phosphatidylserine externalization. Biochem Soc Trans 2004; 32:477-9.
17. Orozco A F, Jorgez C J, Horne C, Marquez-Do D A, Chapman M R, Rodgers J R, Bischoff F Z, Lewis D E. Membrane protected apoptotic trophoblast microparticles contain nucleic acids: relevance to preeclampsia. Am J Pathol 2008; 173:1595-608.
18. Xia Y, Wen H Y, Kellems R E. Angiotensin II inhibits human trophoblast invasion through AT1 receptor activation. J Biol Chem 2002; 277:24601-8.
19. Montes M, Jaensson E A, Orozco A F, Lewis D E, Corry D B. A general method for bead-enhanced quantitation by flow cytometry. J Immunol Methods 2006; 317:45-55.
20. Tung J W, Parks D R, Moore W A, Herzenberg L A. New approaches to fluorescence compensation and visualization of FACS data. Clin Immunol 2004; 110:277-83.
21. Pattanapanyasat K, Noulsri E, Fucharoen S, Lerdwana S, Lamchiagdhase P, Siritanaratkul N, Webster H K. Flow cytometric quantitation of red blood cell vesicles in thalassemia. Cytometry B Clin Cytom 2004; 57:23-31.
22. Ruggeri Z M. Platelets in atherothrombosis. Nat Med 2002; 8:1227-34.
23. Jorgez C J, Simpson J L, Bischoff F Z. Recovery and amplification of placental RNA from dried maternal blood spots: utility for non-invasive prenatal diagnosis. Reprod Biomed Online 2006; 13:558-61.
24. Jorgez C J, Dang D D, Simpson J L, Lewis D E, Bischoff F Z. Quantity versus quality: optimal methods for cell-free DNA isolation from plasma of pregnant women. Genet Med 2006; 8:615-9.
25. Robert S, Poncelet P, Lacroix R, Arnaud L, Giraudo L, Hauchard A, Sampol J, Dignat-George F. Standardization of platelet-derived microparticle counting using calibrated beads and a Cytomics FC500 routine flow cytometer: a first step towards multicenter studies? J Thromb Haemost 2009; 7:190-7.
26. Jy W, Tiede M, Bidot C J, Horstman L L, Jimenez J J, Chirinos J, Ahn Y S. Platelet activation rather than endothelial injury identifies risk of thrombosis in subjects positive for antiphospholipid antibodies. Thromb Res 2007; 121:319-25.
27. Garcia S, Chirinos J, Jimenez J, Del Carpio Munoz F, Canoniero M, Jy W, Jimenez J, Horstman L, Ahn Y. Phenotypic assessment of endothelial microparticles in patients with heart failure and after heart transplantation: switch from cell activation to apoptosis. J Heart Lung Transplant 2005; 24:2184-9.
28. Toth B, Nieuwland R, Liebhardt S, Ditsch N, Steinig K, Stieber P, Rank A, Gohring P, Thaler C J, Friese K and others. Circulating microparticles in breast cancer patients: a comparative analysis with established biomarkers. Anticancer Res 2008; 28:1107-12.
29. Lok C A, Van Der Post J A, Sargent I L, Hau C M, Sturk A, Boer K, Nieuwland R. Changes in microparticle numbers and cellular origin during pregnancy and preeclampsia. Hypertens Pregnancy 2008; 27:344-60.
30. Le Roy C, Varin-Blank N, Ajchenbaum-Cymbalista F, Letestu R. Flow cytometry APC-tandem dyes are degraded through a cell-dependent mechanism. Cytometry A 2009; 75:882-90.
31. Salameire D, Le Bris Y, Fabre B, Fauconnier J, Solly F, Pernollet M, Bonnefoix T, Leroux D, Plumas J, Jacob M C. Efficient characterization of the TCR repertoire in lymph nodes by flow cytometry. Cytometry A 2009; 75:743-51.
32. Zhao H, Traganos F, Dobrucki J, Wlodkowic D, Darzynkiewicz Z. Induction of DNA damage response by the supravital probes of nucleic acids. Cytometry A 2009; 75:510-9.
33. Bratosin D, Tcacenco L, Sidoroff M, Cotoraci C, Slomianny C, Estaquier J, Montreuil J. Active caspases-8 and -3 in circulating human erythrocytes purified on immobilized annexin-V: a cytometric demonstration. Cytometry A 2009; 75:236-44.
34. Wallace P K, Tario J D, Jr., Fisher J L, Wallace S S, Emstoff M S, Muirhead K A. Tracking antigen-driven responses by flow cytometry: monitoring proliferation by dye dilution. Cytometry A 2008; 73:1019-34.
35. Rimaniol A C, Garcia G, Till S J, Capel F, Gras G, Balabanian K, Emilie D, Humbert M. Evaluation of CD4+ T cells proliferating to grass pollen in seasonal allergic subjects by flow cytometry. Clin Exp Immunol 2003; 132:76-80.
36. Daubeuf S, Bordier C, Hudrisier D, Joly E. Suitability of various membrane lipophilic probes for the detection of trogocytosis by flow cytometry. Cytometry A 2009; 75:380-9.
37. Toth B, Nikolajek K, Rank A, Nieuwland R, Lohse P, Pihusch V, Friese K, Thaler C J. Gender-specific and menstrual cycle dependent differences in circulating microparticles. Platelets 2007; 18:515-21.
38. Daniel L, Fakhouri F, Joly D, Mouthon L, Nusbaum P, Grunfeld J P, Schifferli J, Guillevin L, Lesavre P, Halbwachs-Mecarelli L. Increase of circulating neutrophil and platelet microparticles during acute vasculitis and hemodialysis. Kidney Int 2006; 69:1416-23.
39. Ogata N, Nomura S, Shouzu A, Imaizumi M, Arichi M, Matsumura M. Elevation of monocyte-derived microparticles in patients with diabetic retinopathy. Diabetes Res Clin Pract 2006; 73:241-8.
40. Toth B, Liebhardt S, Steinig K, Ditsch N, Rank A, Bauerfeind I, Spannagl M, Friese K, Reininger A J. Platelet-derived microparticles and coagulation activation in breast cancer patients. Thromb Haemost 2008; 100:663-9.
41. Nantakomol D, Chimma P, Day N P, Dondorp A M, Combes V, Krudsood S, Looareesuwan S, White N J, Pattanapanyasat K, Chotivanich K. Quantitation of cell-derived microparticles in plasma using flow rate based calibration. Southeast Asian J Trop Med Public Health 2008; 39:146-53.
42. Halicka H D, Bedner E, Darzynkiewicz Z. Segregation of RNA and separate packaging of DNA and RNA in apoptotic bodies during apoptosis. Exp Cell Res 2000; 260:248-56.
43. Walker M C, Murphy K E, Pan S, Yang Q, Wen S W. Adverse maternal outcomes in multifetal pregnancies. Bjog 2004; 111:1294-6.
44. Huang W, Yu L F, Zhong J, Qiao M M, Jiang F X, Du F, Tian X L, Wu Y L. Angiotensin II type 1 receptor expression in human gastric cancer and induces MMP2 and MMP9 expression in MKN-28 cells. Dig Dis Sci 2008; 53:163-8.
45. Kosaka T, Miyajima A, Shirotake S, Kikuchi E, Hasegawa M, Mikami S, Oya M. Ets-1 and hypoxia inducible factor-1alpha inhibition by angiotensin II type-1 receptor blockade in hormone-refractory prostate cancer. Prostate 2009.
46. Xu Z G, Miao L N, Cui Y C, Jia Y, Yuan H, Wu M. Angiotensin II type 1 receptor expression is increased via 12-lipoxygenase in high glucose-stimulated glomerular cells and type 2 diabetic glomeruli. Nephrol Dial Transplant 2009; 24:1744-52.
47. Wang G D, Wang X Y, Hu H Z, Fang X C, Liu S, Gao N, Xia Y, Wood J D. Angiotensin receptors and actions in guinea pig enteric nervous system. Am J Physiol Gastrointest Liver Physiol 2005; 289:G614-26.
48. Dimitrijevic I, Malmsjo M, Andersson C, Rissler P, Edvinsson L. Increased angiotensin II type 1 receptor expression in temporal arteries from patients with giant cell arteritis. Ophthalmology 2009; 116:990-6.
49. Suganuma T, Ino K, Shibata K, Kajiyama H, Nagasaka T, Mizutani S, Kikkawa F. Functional expression of the angiotensin II type 1 receptor in human ovarian carcinoma cells and its blockade therapy resulting in suppression of tumor invasion, angiogenesis, and peritoneal dissemination. Clin Cancer Res 2005; 11:2686-94.
50. Polakova K, Bennink J R, Yewdell J W, Bystricka M, Bandzuchova E, Russ G. Mild acid treatment induces cross-reactivity of 4H84 monoclonal antibody specific to nonclassical HLA-G antigen with classical HLA class I molecules. Hum Immunol 2003; 64:256-64.

What is claimed is:

1. A method of enriching microparticles of fetal origin having fetal nucleic acids from a maternal sample, the method comprising the steps of
a) combining in a volume of liquid
i) an antibody that binds to a fetal specific antigen and
ii) a microparticle of fetal origin, wherein the microparticle includes the fetal specific antigen,
b) forming an immunocomplex comprising the microparticles and the antibody, and
c) isolating the immunocomplex from the volume of liquid.

2. The method of claim 1 wherein step b) further comprises forming an immunocomplex comprising a magnetically interacting material and wherein step c) comprises isolating the immunocomplex by application of a magnetic field to at least a portion of the volume of liquid.

3. The method of any one of claims 1-2, wherein the volume of liquid comprises cell free maternal plasma and/or maternal urine.

4. The method of any one of claims 1-3, wherein at least one fetal specific antigen is an HLA-G having an epitope such as the HLA-G epitope recognized by mAb MEM-G/1 or by mAb 2A12.

5. The method of any one of claims 1-3, wherein at least one fetal specific protein is a human placental alkaline phosphatase having an epitope such as the human placental alkaline phosphatase epitope recognized by mAb H17E2.

6. The method of claim 4, wherein at least one antibody is MEM-G/1 or 2A12.

7. The method of claim 4 or 6, wherein the volume of liquid comprises cell free maternal plasma representing a first or second trimester pregnancy.

8. The method of claim 5, wherein at least one antibody is H17E2.

9. The method of claim 5 or 8, wherein the volume of liquid comprises cell free maternal plasma representing a first or second trimester pregnancy.

10. The method of any one of claims 2-9, wherein the magnetically interacting material is a nanoparticle coated with an antibody target, such as dextran, and wherein the immunocomplex comprises a tetrameric antibody complex comprising
a) an antibody to the nanoparticle coat,
b) the antibody to a fetal specific protein, and
c) an antibody which binds to both the antibody to the nanoparticle coat and the antibody to the fetal specific protein.

11. The method of claim 1, wherein the antibody to the fetal specific protein comprises a fluorescent tag and step c) comprises isolating the immunocomplex by flow cytometry, fluorescent activated sorting.

12. The method of claim 11, wherein the fetal specific protein is HLA-G, step b) further comprises forming an immunocomplex comprising the microparticles and fluorescently labeled anti-CD49e and anti-CD51 antibodies, and step c) comprises polychromatic flow cytometry, fluorescent activated sorting to isolate microparticles labeled CD49e+, CD51+ and HLA-G+.

13. The method of any one of claims 1-12, further comprising the step of isolating the fetal origin nucleic acid associated with the microparticle of fetal origin.

14. The method of claim 13, further comprising the step of directly sequencing or amplifying a portion of the nucleic acid associated with the microparticle of fetal origin.

15. The method of claim 14 wherein the amplified of sequenced portion is indicative of the presence or absence of an inherited trait.

16. A method of enriching microparticles of fetal origin having fetal nucleic acids from a maternal sample, the method comprising the steps of
a) combining in a volume of liquid
i) an antibody that binds to a fetal specific antigen and
ii) a microparticle of fetal origin, wherein the microparticle includes the fetal specific antigen,
b) forming an immunocomplex comprising the microparticles and the antibody, and
c) enriching the immunocomplex.

17. A method of detecting a fetal nucleic acid, the method comprising the steps of:
a) enriching a microparticle of fetal origin having a fetal nucleic acid from a maternal sample by combining in a volume of liquid an antibody that binds to a fetal specific antigen and a microparticle of fetal origin, wherein the microparticle includes the fetal specific antigen, wherein the microparticle and the antibody form an immunocomplex; and
b) performing a diagnostic test on the microparticle.

18. The method of claim 17 further comprising the step of:
c) performing a diagnostic test on the fetal nucleic acid within the microparticle.

19. The method of any one of the preceding claims further comprising one or more further purifications of the microparticles.
